(19) United States
(12) Patent Application Publication
(10) Pub. No.: US 2024/0359031 A1
(43) Pub. Date: Oct. 31, 2024

(54) Light Emitting Opthalmic Lens

(71) Applicant: HOYA Optical Labs of America, Inc., Lewisville, TX (US)

(72) Inventors: Michael Marshall, Ramsey, MN (US); Suvagata Tripathy, Ramsey, MN (US); Joshua Miller, Ramsey, MN (US); Jeffrey Brown, Ramsey, MN (US); Griffith Altmann, Ramsey, MN (US)

(73) Assignee: HOYA Optical Labs of America, Inc., Lewisville, TX (US)

(21) Appl. No.: 18/648,134

(22) Filed: Apr. 26, 2024

Related U.S. Application Data
(60) Provisional application No. 63/498,655, filed on Apr. 27, 2023.

(51) Int. Cl. A61N 5/06; G02C 11/00

(52) U.S. Cl. CPC A61N 5/0622; G02C 11/10; A61N 2005/0626; A61N 2005/063; A61N 2005/0648; A61N 2005/0653; A61N 2005/0663

(57) ABSTRACT

A light emitting ophthalmic lens in which a light source is incorporated into a lens. The light source may be configured to emit light within a specific wavelength, such as but not limited to a blue light having a wavelength of about 470 nanometers. The ophthalmic lens may include one or more light guiding elements for directing the light emitted from the light source onto a desired region of an eye, such as the outer areas of the retina. The light source may be attached to the ophthalmic lens or may be integrated therewith. The one or more light guiding elements may be configured to reflect, diffuse, disperse, defocus, and/or diffract the light from the light source towards the eye. A control unit may both power the light source and allow for programming various functions of the light source.

### RELATED APPLICATIONS

This application claims benefit of and priority to U.S. Patent Application Ser. No. 63/498,655 filed Apr. 27, 2023 and entitled Light Emitting Opthalmic Lens, which is hereby incorporated herein by reference in its entirety.

### BACKGROUND OF THE INVENTION

Ocular light therapy using specific wavelengths of light is commonly used to treat a wide range of conditions in patients. As an example, blue light therapy has been found to be useful in treating conditions such as depression and seasonal affective disorder (SAD), and has been shown to potentially improve IQ in some instances.

Ocular light therapy devices may also be useful for addressing a number of other problems, such as, among others, circadian rhythm disruption, jet lag, sleep disorders, shift-work disorders, pre-menstrual syndrome, late luteal phase dysphoric disorder (LLPDD), bulimia and eating disorders, chronic fatigue, migraines, or retinal conditions including retinopathy and retinal vein occlusion, and may further reduce inflammation and improve blood flow to help promote eye tissue repair and healing to help treat eye injuries.

Generally, such ocular light therapy may rely upon handheld or desk-mounted light sources that may be bulky or may be fixed in one location. Such handheld or desk-mounted light sources may restrict a patient's freedom of movement, among various other shortcomings. Other ocular light therapy systems may instead passively filter out certain wavelengths, such as by use of filters which reduce the transmission of undesirable wavelengths while allowing desirable wavelengths to pass through unimpeded.

In view of the above, it is desirable to provide an ocular light therapy system and method which is active rather than passive, which is less bulky, which is portable, and/or which does not restrict freedom of movement of the patient or wearer.

### SUMMARY OF THE INVENTION

Disclosed herein are various example embodiments of a light emitting ophthalmic lens which may include a light source and one or more light guiding elements formed within the lens body.

In an example embodiment, the light source may comprise a blue light source.

In an example embodiment, the light source may comprise a laser emitter.

In an example embodiment, the light source may comprise a light emitting diode.

In an example embodiment, the light source may be configured to emit blue light having a wavelength of about 470 nanometers.

In an example embodiment, the light source may comprise a red light source.

In an example embodiment, the light source may be configured to emit red light having a wavelength of about 640 nanometers.

In an example embodiment, the light source may comprise a green light source.

In an example embodiment, the light source may be configured to emit green light having a wavelength of about 530 nanometers.

In an example embodiment, the light source may comprise a yellow light source.

In an example embodiment, the light source may be configured to emit yellow light having a wavelength of about 580 nanometers.

In an example embodiment, the light source may comprise any wavelength or wavelength range in the visible light spectrum.

In an example embodiment, the light source may be comprised of light emitting semiconductors such as nanocrystals.

In an example embodiment, the light source may be oriented to emit light directly into the eye, such as an outer area of the retina.

In an example embodiment, the light source may be oriented to emit light indirectly into the eye, such as by use of one or more light guiding elements.

In an example embodiment, the light source may be attached to the ophthalmic lens.

In an example embodiment, the light source may be attached to an outer rim of the ophthalmic lens.

In an example embodiment, the light source may be secured within the ophthalmic lens.

In an example embodiment, the light source may be secured to an eyeglasses frame encompassing an ophthalmic lens including one or more light guiding elements.

In an example embodiment, the light source may be oriented to emit light towards one or more light guiding elements.

In an example embodiment, the one or more light guiding elements may be comprised of microlenses.

In an example embodiment, the one or more light guiding elements may be comprised of nanoparticles, microparticles, or other types of light scattering centers.

In an example embodiment, the one or more light guiding elements may comprise openings or divots formed within the ophthalmic lens.

In an example embodiment, the one or more light guiding elements may be grouped near an inner or central area of the ophthalmic lens.

In an example embodiment, the one or more light guiding elements may be grouped near an outer area or an outer perimeter of the ophthalmic lens.

In an example embodiment, the one or more light guiding elements may be distributed uniformly or non-uniformly across an entire surface area of the ophthalmic lens.

In an example embodiment, the one or more light guiding elements may be grouped in two or more areas or regions of the ophthalmic lens.

In some aspects, the techniques described herein relate to a light emitting ophthalmic lens, including: a lens; a light guiding element attached to the lens; and a light source oriented to emit a light towards the light guiding element.

In some aspects, the techniques described herein relate to a light emitting ophthalmic lens, wherein the light is a blue light.

In some aspects, the techniques described herein relate to a light emitting ophthalmic lens, wherein the blue light has a wavelength of between 460 nanometers and 480 nanometers.

In some aspects, the techniques described herein relate to a light emitting ophthalmic lens, wherein the blue light has a wavelength of 470 nanometers.

In some aspects, the techniques described herein relate to a light emitting ophthalmic lens, wherein the light source is configured to emit the light directly into an eye.

In some aspects, the techniques described herein relate to a light emitting ophthalmic lens, wherein the light source is positioned so as to emit light directly onto one or more outer areas of a retina of an eye.

In some aspects, the techniques described herein relate to a light emitting ophthalmic lens, wherein the light source is configured to emit light indirectly into an eye.

In some aspects, the techniques described herein relate to a light emitting ophthalmic lens, wherein the light source is attached to an outer rim of the lens.

In some aspects, the techniques described herein relate to a light emitting ophthalmic lens, wherein the light source is embedded into a body of the lens.

In some aspects, the techniques described herein relate to a light emitting ophthalmic lens, wherein the light source is attached to an inner surface of the lens or an outer surface of the lens.

In some aspects, the techniques described herein relate to a light emitting ophthalmic lens, wherein the light source is a light emitting diode.

In some aspects, the techniques described herein relate to a light emitting ophthalmic lens, wherein the light source is a polymer light emitting diode.

In some aspects, the techniques described herein relate to a light emitting ophthalmic lens, wherein the light source is an organic light emitting diode.

In some aspects, the techniques described herein relate to a light emitting ophthalmic lens, wherein the light source is one or more nanoparticles or microparticles.

In some aspects, the techniques described herein relate to a light emitting ophthalmic lens, wherein the light source is one or more light emitting semiconductor nanocrystals.

In some aspects, the techniques described herein relate to a light emitting ophthalmic lens, wherein the light is a red light.

In some aspects, the techniques described herein relate to a light emitting ophthalmic lens, wherein the light is a green light.

In some aspects, the techniques described herein relate to a light emitting ophthalmic lens, wherein the light is a yellow light.

In some aspects, the techniques described herein relate to a light emitting ophthalmic lens, wherein the light guiding element is embedded within a body of the lens.

In some aspects, the techniques described herein relate to a light emitting ophthalmic lens, wherein the light guiding element is positioned on an outer surface of the lens.

In some aspects, the techniques described herein relate to a light emitting ophthalmic lens, wherein the light guiding element is positioned on an inner surface of the lens.

In some aspects, the techniques described herein relate to a light emitting ophthalmic lens, wherein the light guiding element is microlenses.

In some aspects, the techniques described herein relate to a light emitting ophthalmic lens, wherein the light guiding element is openings formed within a body of the lens.

In some aspects, the techniques described herein relate to a light emitting ophthalmic lens, wherein the light guiding element is divots.

In some aspects, the techniques described herein relate to a light emitting ophthalmic lens, wherein the light guiding element is a laminate having areas of a different refractive index.

In some aspects, the techniques described herein relate to a light emitting ophthalmic lens, wherein the light guiding element is voids formed within a body of the lens.

In some aspects, the techniques described herein relate to a light emitting ophthalmic lens, wherein the light guiding element is a laminate having a grid formed from a plurality of areas of a different refractive index.

In some aspects, the techniques described herein relate to a light emitting ophthalmic lens, further including a frame, wherein the lens is attached to the frame, and wherein the light source is attached to the frame.

In some aspects, the techniques described herein relate to a light emitting ophthalmic lens, wherein the light source is attached to the lens.

In some aspects, the techniques described herein relate to a light emitting ophthalmic lens, further including a control for varying a light intensity of the light source.

In some aspects, the techniques described herein relate to a light emitting ophthalmic lens system, including: a lens; one or more light guiding elements attached to the lens; a light source attached to the lens, the light source being configured to emit a light towards the one or more light guiding elements; and a control unit in electrical communication with the light source.

In some aspects, the techniques described herein relate to a light emitting ophthalmic lens system, wherein the control unit is a power source.

In some aspects, the techniques described herein relate to a light emitting ophthalmic lens system, wherein the power source is a DC power source.

In some aspects, the techniques described herein relate to a light emitting ophthalmic lens system, wherein the control unit is one or more sensors or monitors.

In some aspects, the techniques described herein relate to a light emitting ophthalmic lens system, wherein the control unit is a programmable logic circuit.

In some aspects, the techniques described herein relate to a light emitting ophthalmic lens system, wherein the control unit is a memory.

In some aspects, the techniques described herein relate to a method of providing optical light therapy, including: orienting a light guiding element near an eye; orienting a light source towards the light guiding element; emitting a light from the light source towards the light guiding element; and guiding light from the light guiding element towards the eye.

In some aspects, the techniques described herein relate to a method, wherein the light is a blue light.

In some aspects, the techniques described herein relate to a method, wherein the light is a red light.

In some aspects, the techniques described herein relate to a method, wherein the light is a green light.

In some aspects, the techniques described herein relate to a method, wherein the light is a yellow light.

In some aspects, the techniques described herein relate to a method, wherein the light is guided by the light guiding element towards an outer area of a retina of the eye.

In some aspects, the techniques described herein relate to a method, wherein the light is guided by the light guiding element towards a central area of a retina of the eye.

In some aspects, the techniques described herein relate to a method, wherein the light is guided by the light guiding element towards an inner area of a retina of the eye.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 is a side view of a light emitting ophthalmic lens in accordance with an example embodiment.

FIG. 2 is a side view of a light emitting ophthalmic lens in accordance with an example embodiment.

FIG. 3 is a side view and close-up view of a light emitting ophthalmic lens in accordance with an example embodiment.

FIG. 4 is a side view of a light emitting ophthalmic lens in accordance with an example embodiment.

FIG. 5 is a block diagram illustrating a control unit for a light emitting ophthalmic lens in accordance with an example embodiment.

FIG. 6 is a front view of a light emitting ophthalmic lens in accordance with an example embodiment.

FIG. 7A is a front view of a light emitting ophthalmic lens in accordance with an example embodiment.

FIG. 7B is a front view of a light emitting ophthalmic lens in accordance with an example embodiment.

FIG. 7C is a front view of a light emitting ophthalmic lens in accordance with an example embodiment.

FIG. 8 is a graph illustrating irradiance of a light emitting ophthalmic lens in accordance with an example embodiment.

FIG. 9 is a front view of an eyeglass frame including light emitting ophthalmic lenses in accordance with an example embodiment.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

For the purposes of this specification, use of the terms “about”, “around”, or “approximately” when referring to a value may be understood to mean within 5% of the stated value (either greater or lesser), inclusive.

Disclosed herein are various embodiments of a light emitting ophthalmic lens which may be used, e.g., for providing blue light therapy to an eye of a patient. The lens may include a light source and one or more light guiding elements, which may function to reflect, diffuse, defocus, disperse, and/or diffract light into or towards the eye when the lens is worn. Alternatively, the lens may include a light source which emits light directly towards the eye.

The ophthalmic lens may be comprised of various lens materials known in the art, including but not limited to polycarbonates, glasses, plastics, Trivex, and the like, including, but not limited to polycarbonate, polyethylene terephthalate (PET) or triacetate cellulose (TAC) laminates. The ophthalmic lens may be formed using various methods known in the art for formation of lenses, including, but not limited to injection molding or casting.

Various types of light sources may be utilized with the light emitting ophthalmic lens. By way of example and without limitation, such light sources may include, e.g., light emitting diodes (LED), organic light emitting diodes (OLED), polymer light emitting diodes (PLED), nano particles, light emitting semiconductor nanocrystal materials, various light emitters such as lasers, and the like.

Various wavelengths of light may be emitted by the light source in different embodiments. For example, the wavelengths of light emitted by the light source may be any wavelength within or any wavelength range within the visible light spectrum, such as between, but not limited to, about 380 nanometers and about 750 nanometers. In one example, the light source may be configured to emit blue light having a wavelength between about 450 nanometers and about 495 nanometers, or more specifically between about 460 nanometers and about 480 nanometers. In a specific example, the light source may be configured to emit blue light at about 470 nanometers within a less than one percent deviation in spectra.

It is noted that blue light at about 470 nanometers has been shown to be very important for setting circadian rhythms and eye physiology and, in some circumstances, blue light having a wavelength of less than 460 nanometers may be problematic for good eye health. Exemplary benefits of ocular light therapy using blue light are shown and described in U.S. Pat. No. 9,138,595, which is hereby incorporated by reference in its entirety. In other examples, the benefits of targeted ocular light therapy using blue light may include alleviating the symptoms of depression and anxiety, such as by stimulating the production of serotonin, and helping to regulate the sleep-wake cycle, such as by promoting wakefulness and suppressing melatonin at certain times of day.

In further examples, the light source may be configured to emit red light, such as, but not limited to, having a wavelength between about 620 nanometers and about 750 nanometers for various health applications or treatments. In more specific examples, the light source may be configured to emit red light at about 640 nanometers, or at about 680 nanometers, within a less than one percent variation in spectra. The benefits of targeted ocular light therapy using red light may include any purpose known in the art, such as, among others, protecting vision over time and supporting the reversal of disorders such as macular degeneration or glaucoma, diabetic retinopathy and retinal vein occlusion, as well as promoting healing in response to various eye injuries by improving blood flow and reducing or suppressing inflammation. Red light ocular therapy may also help to improve color contrast sensitivity and rod sensitivity within the eyes, and thus may help some see more vivid colors or improve vision in low light environments.

In still further examples, the light source may be configured to emit green light, such as, but not limited to, between about 510 nanometers and about 570 nanometers. In one specific example, the light source may be configured to emit green light at 520 nanometers or 550 nanometers within about a one percent variation in spectra. The benefits of targeted ocular light therapy using green light may include any purpose known in the art, such as, among others, reducing the severity and frequency of migraine headaches, improving various conditions associated with neuropathic or chronic pain, and reducing or alleviating the symptoms of depression and anxiety, or improving the ability to sleep, by having a calming and relaxing effect on the nervous system.

In additional examples, the light source may be configured to emit yellow light, such as, but not limited to, between about 570 nanometers and about 590 nanometers. In one specific example, the light source may be configured to emit yellow light at 580 nanometers within about a one percent variation in spectra. The benefits of targeted ocular light therapy using yellow light may be used for any purpose known in the art, such as, among others, helping to promote relaxation and improve sleep quality by stimulating the production of melatonin, helping to alleviate neuropathic or chronic pain, and promoting healing in response to various eye injuries by reducing or suppressing inflammation. In still further examples, the light source may emit light including a combination of two or more different colors, such as to help increase the effectiveness of some treatments or treatment plans. The light emitting ophthalmic lens may rely upon direct light, in which a light source is oriented to direct light straight onto one or more portions of the eye, or indirect light, in which a light source is oriented to direct light to one or more light guiding or light scattering elements, which then reflect, diffuse, defocus, disperse, and/or diffract light onto one or more portions of the eye. Thus, the light source may be attached to an outer surface of the ophthalmic lens or may be embedded or integrated directly into the lens body. In other examples, the light source may be attached to or embedded completely, or partially, within a device external to the lens, such as, but not limited to, an eyeglass frame capable of receiving or encompassing one or more light emitting ophthalmic lenses.

The light source may be attached, either directly or indirectly, to an outer surface of the ophthalmic lens. As a first example, the light source may be attached to an outer rim of the lens, with the light source being completely external to the lens or being at least partially embedded in the outer rim of the lens. As a second example, the light source may be attached to an inner surface of the lens, in which case the light may be emitted directly into the eye without any light piping or guidance. As a third example, the light source may be attached to an outer surface of the lens, in which case the lens material itself may transmit the light and thus light piping may be present. However, the transmission of the light may be impacted if any laminates are utilized on the lens, or by any adhesives utilized to secure such laminates to the lens. As a fourth example, the light source may be integrated or embedded directly into the body of the lens itself. Such a configuration may allow light piping to occur through the lens material while bypassing any laminates and/or adhesives.

Where the light source is oriented or positioned for application of indirect light, one or more light guiding elements may be formed in or attached to the lens to aid in reflecting, diffusing, dispersing, defocusing, scattering, and/or diffracting light into or towards the eye. Such light guiding elements may include, e.g., use of a different index of refraction embedded onto a laminate, microlenses, openings such as divots, laser ablation of portions of the lens, and the like.

Where the light source is oriented or positioned for application of direct light, the light source may be embedded or integrated directly into the lens. In one example, light emitting semiconductor nanocrystals may be embedded or integrated within the lens to direct light toward or into the eye. In another example, light emitting nanoparticles or microparticles may be embedded or integrated within the lens to direct light toward or into the eye.

In view of all the above, it is to be appreciated that different colors or wavelengths of light or combinations thereof may be helpful in treating various ocular or health issues. It is further to be appreciated that these issues may require different treatment plans that could vary in light color, light intensity, duration of treatment, or frequency of treatment, and that individual physical constitutions and responses to light therapy may vary widely and require individual tailoring. Thus, it is important that the light source and a control system therefore may be configured to allow for the customization and adjustment of these factors.

In this regard, the manner by which the light source is powered and/or programmed may vary. A control system including a programmable controller may allow for the power level or the brightness of the light source to be adjusted as needed. As may be appreciated, this may allow a user to selector otherwise vary the intensity or color of the light provided by the light source for various reasons, such as to help improve user comfort and/or to correspond to, or otherwise comply with, a recommended or self-generated treatment plan. The control system may also include a power source, such as a battery, and an on/off switch for activating or deactivating the light source. The control system may additionally include a programmable timer so that the light source may be activated or deactivated in preset cycles. The control system may also include memory for storing various operating parameters or programming. The control system may further include a monitor or sensor, with the readings of the monitor or sensor being stored in the memory.

Specific example embodiments are described further below. However, it should be understood that any of the features from any of the embodiments can be mixed and matched with each other in any combination. Hence, the present invention should not be restricted to only these embodiments, but any broader combination thereof.

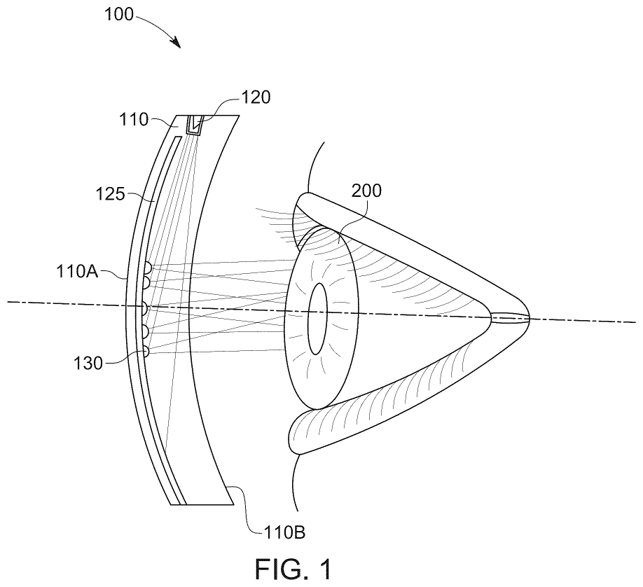

FIG. 1 is a side view of a light emitting ophthalmic lens 100, in accordance with an example embodiment. As shown in in FIG. 1, one example of the light emitting ophthalmic lens 100 may comprise an ophthalmic lens 110 having an outer surface 110A facing away from the eye 200 and an inner surface 110B facing towards the eye 200. A light source 120 may be attached at or near an outer rim of the ophthalmic lens 110. The light source 120 may be completely external of the ophthalmic lens 110, completely internal of the ophthalmic lens 110, or partially external and partially internal to the ophthalmic lens 110. In further examples, the light source 120 may be affixed or otherwise attached to the outer surface 110A, the inner surface 110B.

One or more light guiding elements 130 may be integrated within the ophthalmic lens 110 body and positioned so as to direct light from the light source 120 towards one or more desired locations on the eye 200, such as, but not limited to, outer areas of the retina, inner areas or a central area of the retina, or substantially or completely across an entire surface area of the eye 200 or the retina thereof.

Continuing to reference FIG. 1, it can be seen that the one or more light guiding elements 130 may be incorporated into a body of the ophthalmic lens 110 itself. While FIG. 1 illustrates that the one or more light guiding elements 130 may include multiple light guiding elements, it should be appreciated that the one or more light guiding elements 130 may include only a single light guiding element in some example embodiments. Further, the number, positioning, and orientation of the one or more light guiding elements 130 illustrated in the example embodiment shown in FIG. 1 should not be construed as limiting in scope, as the number, positioning, and orientation of the one or more light guiding elements 130 may vary in different embodiments to suit different examples of the ophthalmic lens 110 which vary in structure or intended application.

In some such examples, the one or more light guiding elements 130 may dispersed, clustered at, or positioned within one or more predefined regions in the ophthalmic lens 110 where each particle acts as a light guide or a light tube. In another example, the one or more light guiding elements 130 may dispersed uniformly, or non-uniformly, throughout or across a partial, or an entire, surface area of the ophthalmic lens 110. In such examples, each of the one or more light guiding elements 30 may be a scattering particle, such as, but not limited to, a micro or nano particle, that acts as a light scattering or deflecting center rather than a light guide.

In any example of the present disclose described above or below, the one or more light guiding elements 130 may include, for example, but not limited to, between about one light guiding element and about ten light guiding elements, between about eleven light guiding elements and about one hundred light guiding elements, or between about one hundred and about one thousand light guiding elements, or still greater numbers of light guiding or scattering elements.

With further reference to FIG. 1, the one or more light guiding elements 130 may comprise one or more laminates 125 which are manufactured into the ophthalmic lens 110 at different areas. These areas may be configured to reflect and diffuse the light from the light source 120 into the eye 200. For example, the one or more laminates 125 may be patterned with a reflection surface in a grid-like or a lattice-like barrier to reflect light from the light source 120 into the eye 200 as shown in FIG. 1. In further examples, the one or more laminates 125 may be pattered with other types or styles of reflection elements or surfaces, such as any random or repeating pattern that may be configured to reflect, diffuse, scatter, or otherwise redirect light from the light source 120 into the eye 200.

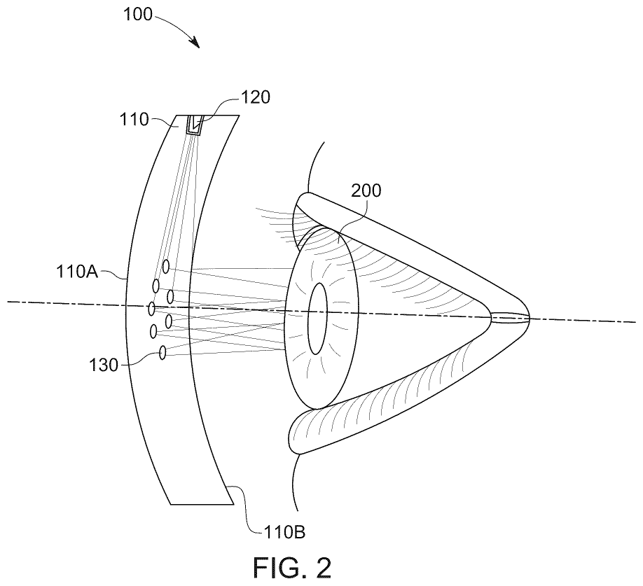

FIG. 2 is a side view of the light emitting ophthalmic lens 100, in accordance with an example embodiment. As shown in FIG. 2, one example of the light emitting ophthalmic lens 100 may be an embodiment in which the one or more light guiding elements 130 may be comprised of, or may include, a number of dispersion points formed in the ophthalmic lens 110, such as, but not limited to, by laser ablation. In such an embodiment, the light source 120 may be attached to, or integrated completed or partially into, an outer rim of the ophthalmic lens 110 as shown in FIG. 2. Such laser ablation can be in an array pattern, or in other sequences, any of which may cause the light from the light source 120 to disperse, diffuse, or deflect toward the eye 200 and thus be seen by the wearer of the ophthalmic lens 110. In one example embodiment, the lens material may function similar to a light pipe, with the laser ablation causing voids or changes in index of refraction capable of causing reflection, dispersion, or redirection of light. An example lens incorporating different refraction areas is shown and described in U.S. Pat. No. 11,029,540, which is hereby incorporated by reference in its entirety.

It should be appreciated that the position of the light source 120 along the outer perimeter, edge, or rim of the ophthalmic lens 110 may vary in different embodiments, and thus should not be construed as limited by the example embodiments shown in the figures. Thus, while the figures illustrate that the light source 120 may be positioned near the top edge or portion of the ophthalmic lens 110, it should be appreciated that the light source 120 may, alternatively, be positioned near a bottom edge or portion of the ophthalmic lens 110, or at any other position along its outer perimeter.

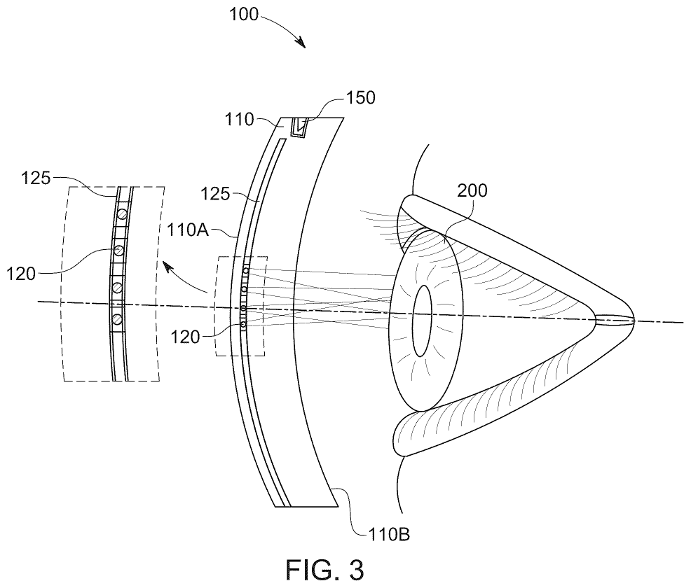

FIG. 3 is a side view and close-up view of the light emitting ophthalmic lens 100, in accordance with an example embodiment. As shown in FIG. 3, one example of the light emitting ophthalmic lens 100 may be an embodiment in which a light source 120 comprises light emitting semiconductor nanocrystals that may be used for either direct or indirect lighting of the eye 200. In the example embodiment shown in FIG. 3, it can be seen that the light source 120 may be realized in the form of a plurality of light emitting semiconductor nanocrystals that are embedded or otherwise integrated into the ophthalmic lens 110 in the vision pathway. However, it should be appreciated that, in some embodiments, such light emitting semiconductor nanocrystals of the light source 120 may be utilized for indirect lighting in combination with the one or more light guiding elements 130.

Examples of light emitting nanocrystal materials are shown and described in U.S. Pat. No. 8,080,437, which is hereby incorporated by reference in its entirety. In such examples, the light source 120 may be formed completely or partially by between about one light emitting semiconductor nanocrystal and about ten light emitting semiconductor nanocrystals, between about eleven light emitting semiconductor nanocrystals and about one-hundred light emitting semiconductor nanocrystals, or between about one-hundred and about one thousand light emitting semiconductor nanocrystals, or still greater numbers of light emitting semiconductor nanocrystals. Moreover, the light emitting semiconductor nanocrystals of the light source 120 may located, position, or grouped in any manner described with respect to the one or more light guiding elements 130 above or below, such as, but not limited to, at any position within the one or more laminates 125, or at any position within other material layers of the ophthalmic lens 110.

Continuing to reference FIG. 3, it can be seen that the one or more laminates 125 may be a conducting, transparent film that may be secured onto, or secured within, the ophthalmic lens 110, such as by use of an optical grade adhesive or the like to secure one or more light emitting semiconductor nanocrystals of the light source 120 in a position for direct or indirect application of emitted light into the eye 200. A power connector 150 may also be incorporated into the ophthalmic lens 110, which may be in electrical communication with the one or more light emitting semiconductor nanocrystals of the light source 120 for powering purposes. By way of example, the power connector 150 may comprise a direct current (“DC”) power connector.

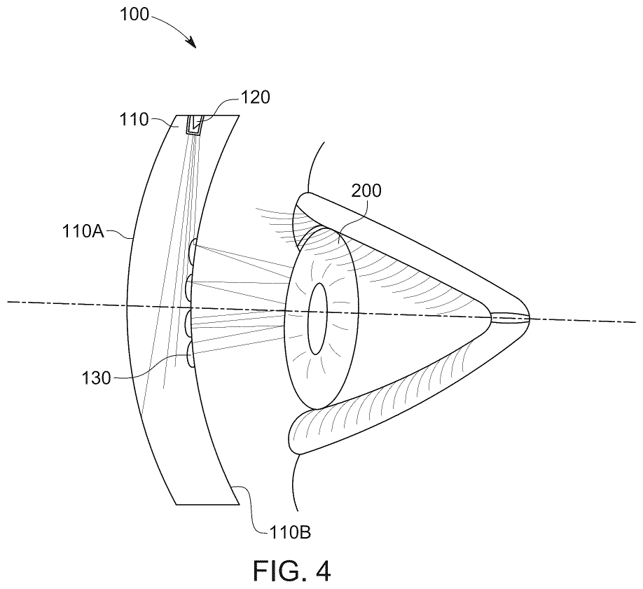

FIG. 4 is a side view of the light emitting ophthalmic lens 100, in accordance with an example embodiment. As shown in FIG. 4, one example of the light emitting ophthalmic lens 100 may be an embodiment in which the light source 120 may be attached or integrated into the ophthalmic lens 110, with the light emitted from the light source 120 being directed to the one or more light guiding elements 130 comprised of one or more microlenses, lenselets, or defocus incorporated multiple segments (“DIMS”). Examples of such microlenses are shown and described in U.S. Pat. Nos. 10,386,654 and 11,131,869, both of which are hereby incorporated by reference in their entireties.

The use of such microlenses may provide the dual function of both providing light therapy from the light source 120 and treating myopia, hyperopia, or inhibiting the progression of or helping to improve other types of ametropia. For example, microlenses may be designed to correct peripheral defocus, and thereby possibly slow the progression of myopia or other types of ametropia progression. In another example, microlenses with peripheral plus power (peripheral hyperopia) may help slow down myopia or other types of ametropia progression. In a further example, microlenses may help modulate the focusing ability of the eye by controlling accommodation (e.g., the eye's ability to focus on objects at different distances), such microlenses may help manage myopia or other types of ametropia progression.

Continuing to reference FIG. 4, it can be seen that the one or more light guiding elements 130 may be positioned at or near an inner surface 110B of the ophthalmic lens 110 facing the eye. However, it should be appreciated that the one or more light guiding elements 130, such as microlenses, shown in FIG. 4 may be positioned at various other locations along or within the ophthalmic lens 110. In any of the example ophthalmic lens 110 shown in FIGS. 1-4, the inner surface 110B may be part of a first lens layer and the outer surface 110A may be part of a second lens layer of the ophthalmic lens 110.

In some such examples, each of these first and the second lens layers may be single polycarbonate layers. In other examples, these first and second lens layers can each be made from multiple layers of similar, or different, materials. Such materials may include, but not limited to polycarbonate sheets or laminates, glass sheets or glass laminates, nylon sheets or nylon laminates, polyimide sheets or laminates, polyethylene terephthalate (“PET”) sheets or laminates, biaxially oriented PET triacetate laminates, or any other non-polarized or polarized sheet laminates.

Further, in any of the above examples, such laminate sheets or other components, such as the one or more laminates 125, microlens, lenselets, defocus incorporated multiple segments, or light emitting nanocrystals may be adhered to one another using various optical adhesives. In one example, a polyurethane adhesive made from reacting V03 and V04 may be used. In other examples, any of pressure sensitive adhesives, moisture cured adhesives, or adhesives cured by ultraviolet light or electron-beams may be used. One specific example, 3M 8213 OCA adhesive curable at about eighty degrees Fahrenheit may be used. Other non-limiting examples may include 3M 8146-2 OCA and 3M CEF 3104AS OCA, which are cured by the application of pressure.

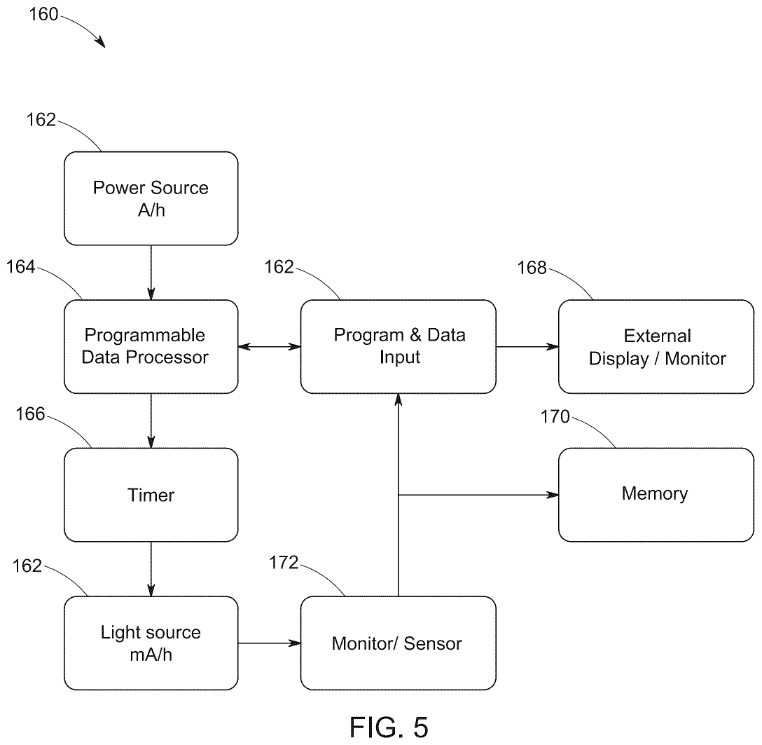

In some examples, the total thickness of the light emitting ophthalmic lens 100 may be between about 10 millimeters and about 40 millimeters. In one example, the first lens layer, which may form the inner surface 110B, may have a thickness measuring about 12 millimeters or about 15 millimeters, and the second lens layer, which may form the outer surface 110A, may have a thickness measuring about 15 millimeters or about 12 millimeters, respectively. FIG. 5 is a block diagram illustrating a control unit 160 for the light emitting ophthalmic lens 100, in accordance with an example embodiment. As shown in FIG. 5, one example of the control unit 160 may be an embodiment in which the control unit 160 may provide multiple functions, including, but not limited to, powering the light source 120, programming the light source 120, storing or executing operation parameters, sensing or monitoring the light source 120, and the like.

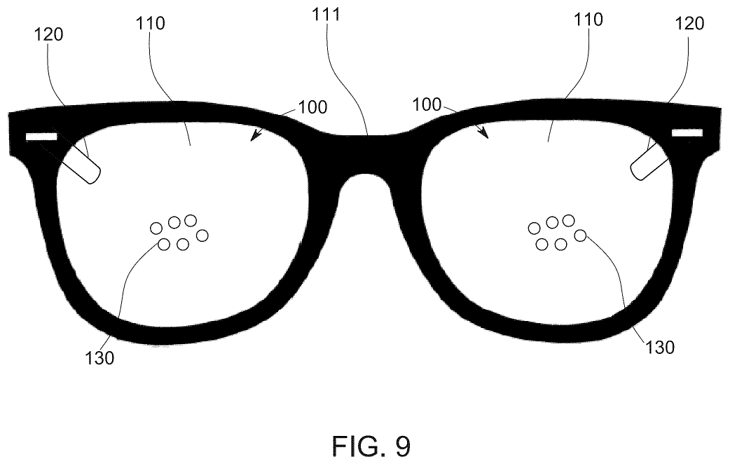

As shown in FIG. 5, the control unit 160 may comprise a power source 162, a programmable data processor 164, a timer 166, an external display or monitor 168, a memory 170, and/or a monitor/sensor 172 for performing a wide range of functions. The control unit 160 may be integrated into the ophthalmic lens 110, or may be electrically connected to or otherwise in electrical communication with the light source 120, such as while remaining external to the ophthalmic lens 110. In one example, the control unit 160 may be completely or partially integrated into the eyeglass frame 111 (FIG. 9). The power source 162 may be rechargeable. By way of example, the light source 120 may require about 0.384 mA/h power consumption which, when incorporating loss in electronics with light emitting diode (“LED”) lighting, may provide about 0.864 mA/h of operational treatment of 60 minutes per day with daily recharging.

In view of the above, the control unit 160 may be a control system capable of enabling a user to program, control, or adjust, such as via one or more user inputs to the programmable data processor 164, various output aspects of the light source 120 including, among others, the power level (e.g., light brightness or intensity), the light color (e.g., wavelength), or the period or duration of light emittance from the light source 120 as needed. In some examples, such user inputs may to an input/output (“I/O”) device of the control unit 160, such as, but not limited to, the external display or monitor 168. In other examples, the I/O device may be a button, switch, or another feature of the programmable data processor 164, the power source 162, or any other component of the control unit 160. In some examples, at least one I/O device may be located on the eyeglass frame 111 shown in FIG. 9, such as to enable to, among others, a user to power-on or power-off the power source 162 or adjust any other output aspect of the light source 120.

In further examples, output aspects of the light source 120 may be customized via other user-input means, such as via a wireless connection with the control unit 160. In some such examples, it is to be appreciated that the control unit 160 may be configured to enable program or data instructions to be transmitted or received over a wireless communication network using a transmission medium, such as via a network interface device of the control unit 160, utilizing any one of a number of transfer protocols including, but not limited to frame relay, internet protocol (IP), transmission control protocol (TCP), user datagram protocol (UDP), hypertext transfer protocol (HTTP), NFC Forum Handover Protocol (NHP), NFC Data Exchange Protocol (NDP), or others. Example wireless communication networks can include a local area network (LAN), a wide area network (WAN), a packet data network (e.g., the Internet), mobile telephone networks (e.g., cellular networks), wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks.

In additional examples, the control unit 160 may be configured to enable program or data instructions to be transmitted or received through wired means, such as through a wired connection with the control unit 160 established via one or more computer data ports (e.g., USB, SATA, etc.) thereof.

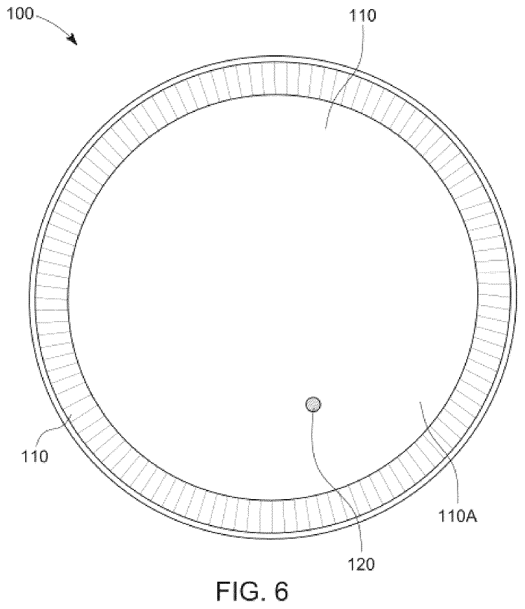

FIG. 6 is a front view of the light emitting ophthalmic lens 100, in accordance with an example embodiment. As shown in FIG. 6, one example of the light emitting ophthalmic lens 100 may be an embodiment in which the light source 120 is incorporated, such as by embedding or laminating, into a body of the ophthalmic lens 110 for direct lighting of the eye. Moreover, the light source 120 including any of one or more active light emitting components thereof, when embedded into the ophthalmic lens 110 for direct light emission, may be located or positioned in any manner described with respect to the one or more light guiding elements 130 above or below, such as, but not limited to, at any position within the one or more laminates 125, or at any position within other material layers of the ophthalmic lens 110.

While not shown, various embodiments may rely upon leads, such as copper leads or the like, to connect the light source 120 when embedded within the ophthalmic lens 110, to an external or attached power source. Such electrical leads may be taped or otherwise secured to various portions of the ophthalmic lens 110, such as, but not limited to, the edge of the ophthalmic lens 110. The use of the light source 120 embedded within the ophthalmic lens 110 may be useful for direct lighting of the eye 200.

The positioning of the light source 120 when embedded within the ophthalmic lens 110 may vary, such as with the light source 120 being embedded at or near the outer surface 110A, the inner surface 110B, or within an internal body or central area of the ophthalmic lens 110. The positioning of the light source 120, when embedded within the ophthalmic lens 110, may have an effect on whether or not light piping occurs or is effective. Further, the positioning of the light source 120 with respect to the eye 200 may also vary depending on the ocular treatment being performed, and as well as other considerations. By way of example, the light source 120 may be embedded in a position to direct light towards an outer area, or alternately an inner or central area, of the retina of the eye 200 (FIGS. 1-4).

More specifically, if a more intensive treatment, or more efficient light path to the eye 200 is desired, any of the one or more light guiding elements 130 or the light source 120 discussed above or below may be configured to directly (e.g., emit) or indirectly (e.g., reflect or redirect) guide light towards an outer area (e.g., peripheral retina) of the eye 200 where rod cells predominate and the eye 200 may be more sensitive to changes in light intensity. Alternatively, if a less intensive treatment, or a less efficient light path to the eye 200 is desired, any of the one or more light guiding elements 130 or the light source 120 discussed above or below may be configured to directly (e.g., emit) or indirectly (e.g., reflect or redirect) guide light towards an inner or central area of the eye 200 (e.g., macula and fovea), where cone cells predominate and they eye 200 may be less sensitive to light intensity.

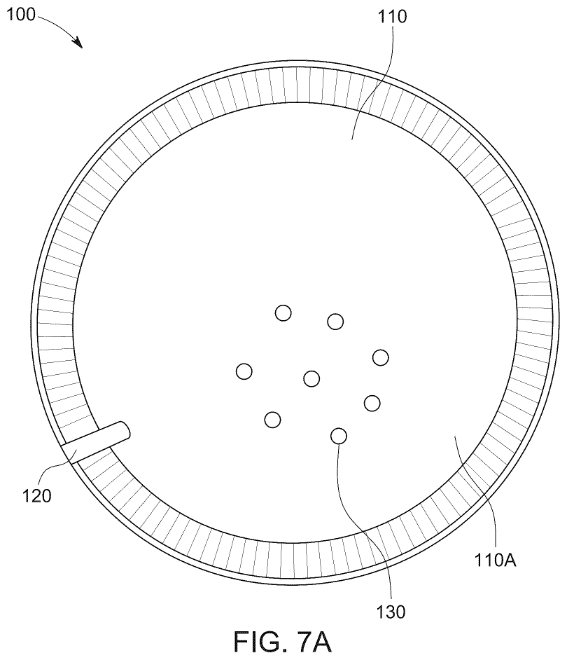

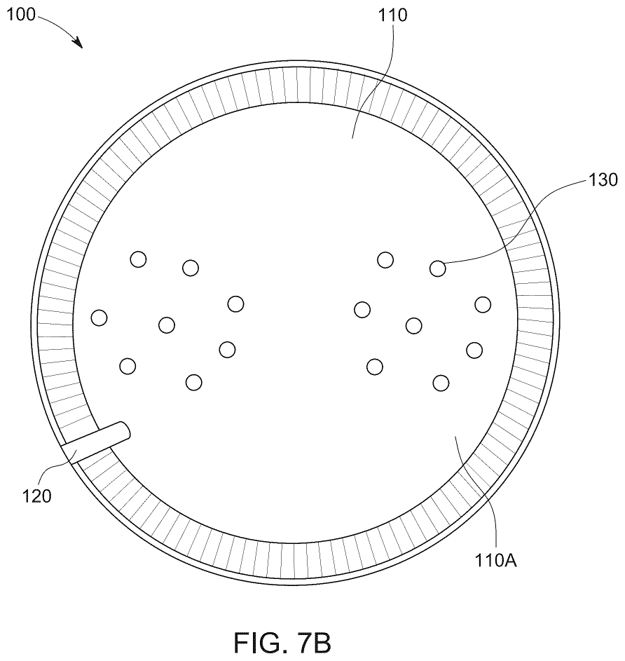

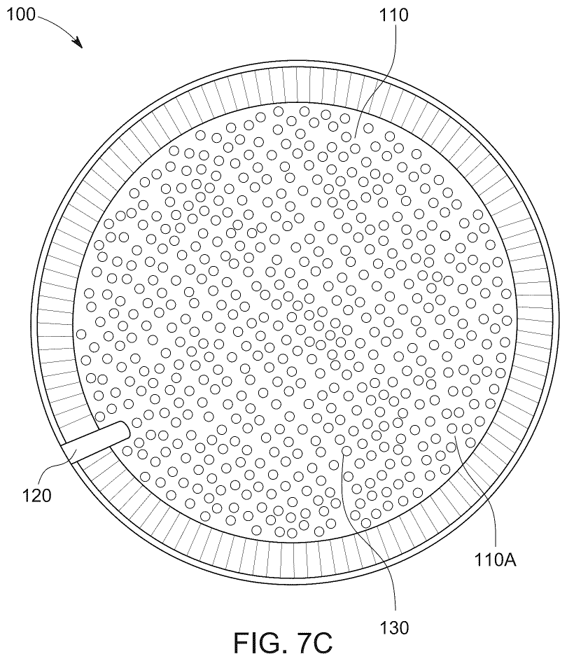

FIGS. 7A-7C are front views of the light emitting ophthalmic lens 100, in accordance with an example embodiment. FIGS. 7A-7C are discussed below concurrently. As shown in FIGS. 7A-7C, some examples of the light emitting ophthalmic lens 100 may be embodiments in which the light source 120 is secured to, or is embedded completely or partially embedded within, the outer rim of the ophthalmic lens 110 and in which the one or more light guiding elements 130 are comprised of small lens dibs which may be drilled directly into the lens to reflect and diffuse light from the light source 120 into the eye 200 (FIGS. 1-4).

Such a configuration may be considered the inverse to the microlens configuration previously shown in, and discussed with respect to, FIG. 4. In some examples, the lens dibs forming the one or more light guiding elements 130 may comprise a diameter of about 1 millimeter and a depth of less than 0.1 millimeter. In some examples, an injection molded lens 100 may be drilled with a hole having a diameter of about 3 millimeters at a depth of about 4 millimeters on the center edge of the lens 100. In some examples, such as shown in FIG. 7A, the one or more light guiding elements 130 may be positioned or grouped near a central or inner area of the ophthalmic lens 110, such as to only, or primarily, guide light toward a central or inner area (e.g., macula and fovea) of the eye 200 (FIGS. 1-4). In such examples, it is to be appreciated that a central or inner area of the ophthalmic lens 110 can be defined as closer to a center point of the ophthalmic lens 110 than to a perimeter edge of the ophthalmic lens 110.

In another example, such as shown in FIG. 7B, the one or more light guiding elements 130 may be positioned or grouped near an outer area of the ophthalmic lens 110, such as to only, or primarily, guide light toward an outer area (e.g., peripheral retina) of the eye 200. In some examples, such as also shown in FIG. 7B, the one or more light guiding elements 130 can be positioned to define two or more individual groups, or predefined regions or areas, that each include various numbers of individual light guiding elements. In one such example, each of the one or more groups may be located closer to the edges of the ophthalmic lens 110 than to the center of the ophthalmic lens 110, as shown in FIG. 7B.

In an additional example, such as shown in FIG. 7C, the one or more light guiding elements 130 may be distributed entirely, or substantially, across a surface area of the ophthalmic lens 110. In such examples, the one or more light guiding elements 130 may be distributed uniformly or non-uniformly (e.g., randomly), and may also be grouped to define, for example, but not limited to, one, two, three, four, five, six, or greater numbers of individual groups, or predefined regions or areas, that each include one or more of the one or more light guiding elements 130. The pattern and positioning of the lens dibs shown in FIGS. 7A-C are merely for exemplary purposes, and thus should not be construed as limiting in scope.

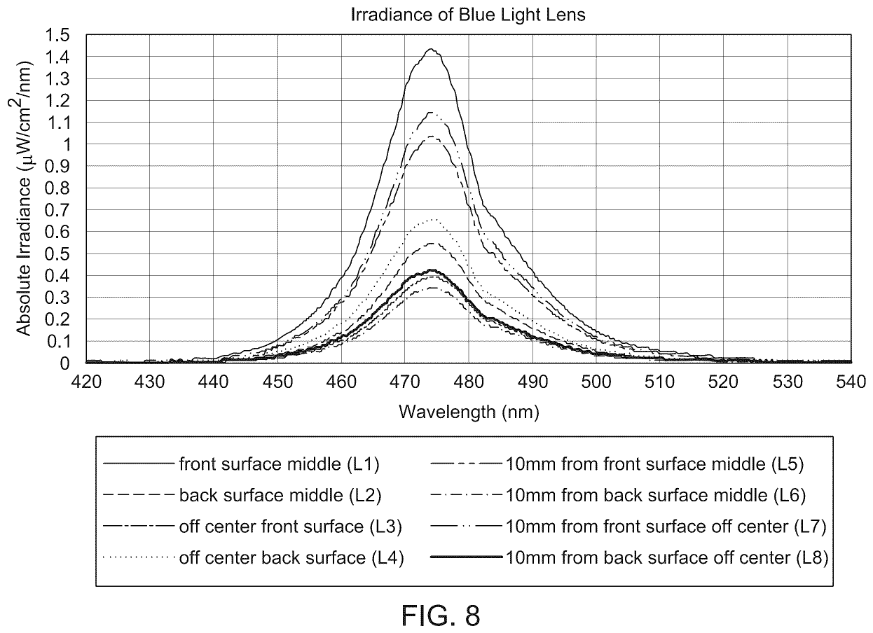

FIG. 8 is a graph illustrating absolute irradiance as a function of wavelength based on different positioning of the one or more light guiding elements 130 and/or the light source 120. An optimal value may comprise about 1 microwatt per cm² per nanometer. As previously noted above, the positioning of the light source 120 (FIGS. 1-4, 6-7, and 9) and the one or more light guiding elements 130 (FIGS. 1-4, 6-7, and 9) may have an effect on the resulting efficiency of light direction or light guidance to the eye 200 (FIGS. 1-4) from the ophthalmic lens 110 (FIGS. 1-4, 6-7, and 9).

In the examples shown in FIG. 8 and represented by lines L1-L8, the light source 120 is at least partially inset into the ophthalmic lens 110 in a location near the inner surface 110B and extends at least partially into an outer rim or edge of the ophthalmic lens 110.

Line L1 represents an observed absolute irradiance value from the ophthalmic lens 110 when the one or more light guiding elements 130 are at the inner surface 110B and are positioned at a center point or central area of the ophthalmic lens 110.

Line L2 represents an observed absolute irradiance value from the ophthalmic lens 110 when the one or more light guiding elements 130 are at the outer surface 110A and are positioned at a center point or central area of the ophthalmic lens 110.

Line L3 represents an observed absolute irradiance value from the ophthalmic lens 110 when the one or more light guiding elements 130 are at the inner surface 110B and are positioned within an inner area or region of the ophthalmic lens 110.

Line L4 represents an observed absolute irradiance value from the ophthalmic lens 110 when the one or more light guiding elements 130 are at the outer surface 110A and are positioned within an inner area or region offset from a center point of the ophthalmic lens 110. Such an inner area or region may be generally defined as a lateral offset from the center point that is closer to the center point than to an outer rim or edge of the ophthalmic lens 110, such as between, but not limited to, about 2 millimeters and about 10 millimeters offset from the center point or central area.

Line L5 represents an observed absolute irradiance value from the ophthalmic lens 110 when the one or more light guiding elements 130 are inset into the ophthalmic lens 110 by about 10 millimeters from the inner surface 110B and are positioned at a center point or central area of the ophthalmic lens 110.

Line L6 represents an observed absolute irradiance value from the ophthalmic lens 110 when the one or more light guiding elements 130 are inset into the ophthalmic lens 110 by about 10 millimeters from the outer surface 110A and are positioned at a center point or central area of the ophthalmic lens 110.

Line L7 represents an observed absolute irradiance value from the ophthalmic lens 110 when the one or more light guiding elements 130 are inset into the ophthalmic lens 110 by about 10 millimeters from the inner surface 110B and are positioned within an inner area or region offset from a center point of the ophthalmic lens 110.

Line L8 represents an observed absolute irradiance value from the ophthalmic lens 110 when the one or more light guiding elements 130 are inset into the ophthalmic lens 110 by about 10 millimeters from the outer surface 110A and are positioned within an inner area or region offset from a center point of the ophthalmic lens 110. Such an inner area or region may be generally defined as a lateral offset from the center point that is closer to the center point than to an outer rim or edge of the ophthalmic lens 110, such as between, but not limited to, about 2 millimeters and about 10 millimeters offset from the center point or central area.

FIG. 9 is a front view of a frame 111 including a pair of light emitting ophthalmic lenses 110, in accordance with an example embodiment. Each of the pair of light emitting ophthalmic lenses 110 may represent any example or embodiment of the light emitting ophthalmic lens 100 discussed above or below. In some examples, the frame 111 may only support a single lens 110.

Additionally, each ophthalmic lens 110 of the pair of light emitting ophthalmic lenses 110 may be similar or different to each other, such as depending upon the needs of the individual patient. In one example, each of the pair of light emitting ophthalmic lenses 110 may be different to each other by virtue of including differently configured microlenses having, or by being shaped or formed to impart, different corrective powers such as, among others, power (“PWR”), which refers to a positive or a negative number indicating the degree of correction for improving near vision and/or distance vision, cylinder (“CYL”), which refers to a positive or negative number for addressing astigmatism, axis (“AX”), addition (“ADD”), single vision near (“SVN”), or single vision distance (“SVD”) using any lens formation process known in the art.

In another example, one of the lenses 110 may include light guiding elements 130 and one of the lenses 110 may not include light guiding elements 130. In another example, one of the lenses 110 may include a first type of light guiding element 130 and one of the lenses 110 may include a second type of light guiding element 130 which is different from the first type of light guiding element 130.

As shown in FIG. 9, one example of the light source 120 may be an embodiment in which the light source 120 is partially integrated into or embedded within the eyeglass frame 111. In such examples, it is to be appreciated that the light source 120 may include or more light emitting components for each of the pair of ophthalmic lenses 110, each of which may comprise any example of the light source 120 previously discussed above. In some examples, a single light source 120 may emit light towards both lenses 110. In other examples, each of the lenses 110 may have its own dedicated light source 120.

The light source(s) 120 may in some examples be attached to or embedded within the frame 111. In some examples, the light source 120 may extend into, or extend from, each rim 115 of the eyeglass frame 111 toward or into each of the pair of ophthalmic lenses 110. In some examples, the light source 120 may be embedded within the frame 111 and thus be flush or inset with respect to outer surfaces of the frame 111.

In some examples, the light source 120 may be located completely or entirely within the arms, bridge, or any other portion of the eyeglass frame 111. In further examples, the light source 120 may be located completely external of the eyeglass frame 111 and attached thereto so as to emit light towards the pair of ophthalmic lenses 110. In still further examples, the light source 120 may be completely external of both the pair of ophthalmic lenses 110 and the eyeglass frame 111 and located remotely, such as attached to a hat, headband, neckband, shirt, or any other garment or device usable to hold the light source 120 and/or the ophthalmic lens 110 or the pair of ophthalmic lenses 110. In some examples, the light source 120 may be attached to the user directly, such as by an adhesive.

### Claim Bank:

Clause 1. A light emitting ophthalmic lens, comprising: a lens; a light guiding element attached to the lens; and a light source oriented to emit a light towards the light guiding element.

Clause 2. The light emitting ophthalmic lens of clause 1, wherein the light is comprised of a blue light.

Clause 3. The light emitting ophthalmic lens of clause 2, wherein the blue light has a wavelength of between 460 nanometers and 480 nanometers.

Clause 4. The light emitting ophthalmic lens of clause 3, wherein the blue light has a wavelength of 470 nanometers.

Clause 5. The light emitting ophthalmic lens of clause 1, wherein the light source is configured to emit the light directly into an eye.

Clause 6. The light emitting ophthalmic lens of clause 1, wherein the light source is positioned so as to emit light directly onto one or more outer areas of a retina of an eye.

Clause 7. The light emitting ophthalmic lens of clause 1, wherein the light source is configured to emit light indirectly into an eye.

Clause 8. The light emitting ophthalmic lens of clause 1, wherein the light source is attached to an outer rim of the lens.

Clause 9. The light emitting ophthalmic lens of clause 1, wherein the light source is embedded into a body of the lens.

Clause 10. The light emitting ophthalmic lens of clause 1, wherein the light source is attached to an inner surface of the lens or an outer surface of the lens.

Clause 11. The light emitting ophthalmic lens of clause 1, wherein the light source is comprised of a light emitting diode.

Clause 12. The light emitting ophthalmic lens of clause 1, wherein the light source is comprised of a polymer light emitting diode.

Clause 13. The light emitting ophthalmic lens of clause 1, wherein the light source is comprised of an organic light emitting diode.

Clause 14. The light emitting ophthalmic lens of clause 1, wherein the light source is comprised of one or more nanoparticles or microparticles.

Clause 15. The light emitting ophthalmic lens of clause 1, wherein the light source is comprised of one or more light emitting semiconductor nanocrystals.

Clause 16. The light emitting ophthalmic lens of clause 1, wherein the light is comprised of a red light.

Clause 17. The light emitting ophthalmic lens of clause 1, wherein the light is comprised of a green light.

Clause 18. The light emitting ophthalmic lens of clause 1, wherein the light is comprised of a yellow light.

Clause 19. The light emitting ophthalmic lens of clause 1, wherein the light guiding element is embedded within a body of the lens.

Clause 20. The light emitting ophthalmic lens of clause 1, wherein the light guiding element is positioned on an outer surface of the lens.

Clause 21. The light emitting ophthalmic lens of clause 1, wherein the light guiding element is positioned on an inner surface of the lens.

Clause 22. The light emitting ophthalmic lens of clause 1, wherein the light guiding element is comprised of microlenses.

Clause 23. The light emitting ophthalmic lens of clause 1, wherein the light guiding element is comprised of openings formed within a body of the lens.

Clause 24. The light emitting ophthalmic lens of clause 1, wherein the light guiding element is comprised of divots.

Clause 25. The light emitting ophthalmic lens of clause 1, wherein the light guiding element is comprised of a laminate having areas of a different refractive index.

Clause 26. The light emitting ophthalmic lens of clause 1, wherein the light guiding element is comprised of voids formed within a body of the lens.

Clause 27. The light emitting ophthalmic lens of clause 1, wherein the light guiding element is comprised of a laminate having a grid formed from a plurality of areas of a different refractive index.

Clause 28. The light emitting ophthalmic lens of clause 1, further comprising a frame, wherein the lens is attached to the frame, and wherein the light source is attached to the frame.

Clause 29. The light emitting ophthalmic lens of clause 1, wherein the light source is attached to the lens.

Clause 30. The light emitting ophthalmic lens of clause 1, further comprising a control for varying a light intensity of the light source.

Clause 31. A light emitting ophthalmic lens system, comprising: a lens; one or more light guiding elements attached to the lens; a light source attached to the lens, the light source being configured to emit a light towards the one or more light guiding elements; and a control unit in electrical communication with the light source.

Clause 32. The light emitting ophthalmic lens system of clause 31, wherein the control unit is comprised of a power source.

Clause 33. The light emitting ophthalmic lens system of clause 32, wherein the power source is comprised of a DC power source.

Clause 34. The light emitting ophthalmic lens system of clause 31, wherein the control unit is comprised of one or more sensors or monitors.

Clause 35. The light emitting ophthalmic lens system of clause 31, wherein the control unit is comprised of a programmable logic circuit.

Clause 36. The light emitting ophthalmic lens system of clause 31, wherein the control unit is comprised of a memory.

Clause 37. A method of providing optical light therapy, comprising: orienting a light guiding element near an eye; orienting a light source towards the light guiding element; emitting a light from the light source towards the light guiding element; and guiding light from the light guiding element towards the eye.

Clause 38. The method of clause 37, wherein the light is comprised of a blue light.

Clause 39. The method of clause 37, wherein the light is comprised of a red light.

Clause 40. The method of clause 37, wherein the light is comprised of a green light.

Clause 41. The method of clause 37, wherein the light is comprised of a yellow light.

Clause 42. The method of clause 37, wherein the light is guided by the light guiding element towards an outer area of a retina of the eye.

Clause 43. The method of clause 37, wherein the light is guided by the light guiding element towards a central area of a retina of the eye.

Clause 44. The method of clause 37, wherein the light is guided by the light guiding element towards an inner area of a retina of the eye.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A light emitting ophthalmic lens, comprising:
a lens;
a light guiding element attached to the lens; and
a light source oriented to emit a light towards the light guiding element.

2. The light emitting ophthalmic lens of claim 1, wherein the light is comprised of a blue light.

3. The light emitting ophthalmic lens of claim 2, wherein the blue light has a wavelength of between 460 nanometers and 480 nanometers.

4. The light emitting ophthalmic lens of claim 1, wherein the light is comprised of a red light.

5. The light emitting ophthalmic lens of claim 1, wherein the light is comprised of a green light.

6. The light emitting ophthalmic lens of claim 1, wherein the light is comprised of a yellow light.

7. The light emitting ophthalmic lens of claim 1, wherein the light guiding element is oriented so as to emit light directly onto an outer area of a retina of an eye.

8. The light emitting ophthalmic lens of claim 1, wherein the light source is attached to an outer rim of the lens.

9. The light emitting ophthalmic lens of claim 1, wherein the light source is embedded into a body of the lens.

10. The light emitting ophthalmic lens of claim 1, wherein the light source is attached to a surface of the lens.

11. The light emitting ophthalmic lens of claim 1, wherein the light source is comprised of a light emitting diode.

12. The light emitting ophthalmic lens of claim 1, wherein the light source is comprised of a light emitting semiconductor nanocrystal.

13. The light emitting ophthalmic lens of claim 1, wherein the light guiding element is embedded within a body of the lens.

14. The light emitting ophthalmic lens of claim 1, wherein the light guiding element is positioned on a surface of the lens.

15. The light emitting ophthalmic lens of claim 1, wherein the light guiding element is comprised of a microlens.

16. The light emitting ophthalmic lens of claim 1, wherein the light guiding element is comprised of an openings formed within a body of the lens.

17. The light emitting ophthalmic lens of claim 1, wherein the light guiding element is comprised of a laminate having areas of a different refractive index.

18. The light emitting ophthalmic lens of claim 1, wherein the light guiding element is comprised of voids formed within a body of the lens.

19. The light emitting ophthalmic lens of claim 1, further comprising a frame, wherein the lens is attached to the frame, and wherein the light source is attached to the frame.

20. A light emitting ophthalmic lens system, comprising:
a lens;
a plurality of light guiding elements attached to the lens;
a light source attached to the lens, the light source being configured to emit a light towards the plurality of light guiding elements; and
a control unit in electrical communication with the light source.

21. The light emitting ophthalmic lens system of claim 20, wherein the control unit is comprised of a power source.

22. The light emitting ophthalmic lens system of claim 20, wherein the control unit is comprised of a sensor.

23. The light emitting ophthalmic lens system of claim 20, wherein the control unit is operable to adjust an intensity of the light emitted from the light source.

24. A method of providing optical light therapy, comprising:
orienting a light guiding element near an eye;
orienting a light source towards the light guiding element;
emitting a light from the light source towards the light guiding element; and
guiding light from the light guiding element towards the eye.

25. The method of claim 24, wherein the light is selected from the group consisting of a blue light, a red light, a green light, and a yellow light.

26. The method of claim 24, wherein the light is guided by the light guiding element towards an outer area of a retina of the eye.

27. The method of claim 24, wherein the light is guided by the light guiding element towards a central area of a retina of the eye.

28. The method of claim 24, wherein the light is guided by the light guiding element towards an inner area of a retina of the eye.
